(19) United States
(12) Patent Application Publication
(10) Pub. No.: US 2017/0036978 A1
(43) Pub. Date: Feb. 9, 2017

(54) PROCESS FOR THE MANUFACTURE OF TERPHENYL COMPOUNDS

(71) Applicant: SOLVAY SPECIALTY POLYMERS USA, LLC, Alpharetta, GA (US)

(72) Inventors: Chantal LOUIS, Alpharetta, GA (US); David B. THOMAS, Atlanta, GA (US)

(73) Assignee: SOLVAY SPECIALTY POLYMERS USA, LLC, Alpharetta, GA (US)

(21) Appl. No.: 15/304,029

(22) PCT Filed: Apr. 8, 2015
(86) PCT No.: PCT/EP2015/057567

§ 371 Date: Oct. 13, 2016

Related U.S. Application Data
(60) Provisional application No. 61/979,824, filed on Apr. 15, 2014.

(30) Foreign Application Priority Data
May 6, 2014 (EP) 14167125.5

(51) Int. Cl. C07C 37/055

(52) U.S. Cl. CPC C07C 37/0555

(57) ABSTRACT

A process for the manufacture of a terphenyl compound [compound (T), herein after] of formula (T): wherein each of R and R′, equal to or different from each other, are selected from the group consisting of halogen, alkyl, aryl, ether, thioether, ester, amide, imide, alkali or alkaline earth metal sulfonate, alkyl sulfonate, alkali or alkaline earth metal phosphonate, alkyl phosphonate, amine and quaternary ammonium; each of j′ and k, equal to or different from each other, are zero or are an integer from 1 to 4, including the steps of (i) reacting at least one organozinc compound of formula (I) with at least one dihalocompound of formula (II) in the presence of a catalyst compound; wherein Y is selected from the group consisting of a chloride, a bromide, an iodide, an alkanesulfonate or a fluoroalkanesulfonate anion, R2 is selected from selected from the group consisting of C₁-C₁₀-alkyl, C₃-C₁₀-cycloalkyl, C₁-C₁₀-halooalkyl and C₃-C₁₀ halocyclo alkyl, each of Z, equal to or different from each other, are selected from the group consisting of a chloride, a bromide, an iodide, an alkanesulfonate or a fluoroalkanesulfonate anion; (ii) a Bayer-Villiger oxidation and (iii) hydrolysis or alcoholysis.

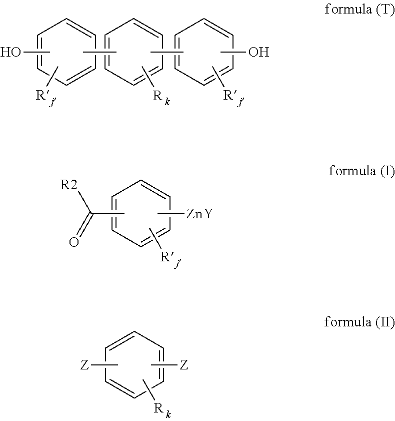

### CROSS-REFERENCE TO RELATED APPLICATIONS

This application claims priority to U.S. provisional application No. 61/979824 filed on 15 April, 2014 and to European application No. 14167125.5 filed on 06 May, 2014, the whole content of each of these applications being incorporated herein by reference for all purposes.

### FIELD OF INVENTION

The present invention relates to a process for the manufacture of terphenyl compounds, in particular 4,4″-dihydroxy-p-terphenyls.

### BACKGROUND OF THE INVENTION

Dihydroxyterphenyls, in particular 4,4″-dihydroxy-p-terphenyls are very useful starting materials in the manufacturing of polymeric materials, in particular polyarylene ether sulfone (PAES) polymers, which are particularly suitable in more demanding, corrosive, harsh chemical, high-pressure and high-temperature (HP/HT) environments, such as notably in oil and gas downhole applications.

Specifically, 4,4″-dihydroxy-p-terphenyls can be prepared by various ways.

4,4″-dihydroxy-p-terphenyl can notably be synthesized by a Kumada coupling of anisole magnesiumbromide and 1,4-dibromobenzene in the presence of a Pd catalyst such as notably described in Y. K. Han, A. Reiser, Macromolecules, 1998, V 31, P 8789-8793 and A. K. Salunke et al, J. Polym. Sci., Part A: Polymer Chemistry, 2002, Vol. 40, 55-69. However, the use of a homogeneous palladium catalyst and of brominated raw materials presents a high cost.

Alternatively, 4,4″-dihydroxy-p-terphenyl can be obtained by tetrazotization of 4,4″-diaminoterphenyl, thereby forming a tetrazotized product. Replacement of the the diazonium groups by hydroxyl groups in an acid environment results in the forming of 4,4″-dihydroxy-p-terphenyl, as notably described by Charles C. Price and George P. Mueller in J. Am. Chem. Soc., 1944, 66 (4), pp 632-634.

Further, U.S. Pat. No. 5,008,472 discloses a process for preparing 4,4″-dihydroxyterphenyl by sulfonation of the terphenyl moiety thereby resulting in the formation of terphenyl-4,4″-disulfonic acid. Caustic hydrolysis of said terphenyl-4,4″-disulfonic acid, which may be in the form of a dialkali metal salt, provides 4,4″-dihydroxyterphenyl.

The disadvantage of these two routes, as described above, is the extremely high cost of the starting raw material p-terphenyl.

EP 0 343 798 A1 describes the preparation of 4,4″ dihydroxy-p-terphenyl, 4-hydroxybiphenyl and related compounds by condensation of cyclic diones or ketones with phenols and dehydrogenation in the presence of a catalyst, in particular Pd/C catalyst and base.

In view of all the above, there is still a current shortfall in the art for an improved process for the manufacture of dihydroxy terphenyl compounds, in particular 4,4″ dihydroxy-p-terphenyl, which can provide dihydroxy terphenyl compounds in high yield, in an efficient manner and by using widely available raw and cheap starting materials and catalysts, and thus suitable to be used as a low cost commercial industrial process.

### DETAILED DESCRIPTION OF THE PRESENT INVENTION

The Applicant has now found that it is possible to advantageously manufacture dihydroxy terphenyl compound, and derivatives thereof, in a very high yield by an easily accessible industrial process, which is advantageously overcoming all the drawbacks of prior art process, as mentioned above.

It is thus an object of the present invention, a process for the manufacture of a terphenyl compound [compound (T), herein after] of formula (T):

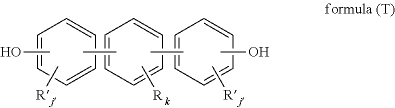

wherein
each of R and R′, equal to or different from each other, are selected from the group consisting of halogen, alkyl, aryl, ether, thioether, ester, amide, imide, alkali or alkaline earth metal sulfonate, alkyl sulfonate, alkali or alkaline earth metal phosphonate, alkyl phosphonate, amine and quaternary ammonium;
each of j′ and k, equal to or different from each other, are zero or are an integer from 1 to 4,
   comprising following steps:

Step 1. reacting at least one organozinc compound of formula (I):

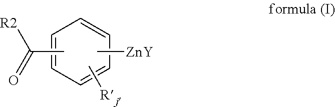

wherein
Y is selected from the group consisting of a chloride, a bromide, an iodide, an alkanesulfonate or a fluoroalkanesulfonate anion, Y is preferably a chloride anion,
R2 is selected from selected from the group consisting of C₁-C₁₀-alkyl, C₃-C₁₀-cycloalkyl, C₁-C₁₀-halooalkyl and C₃-C₁₀ halocycloalkyl,
R′ and j′, have the meanings given above,
   with at least one dihalocompound [compound (HH), herein after] of formula (II):

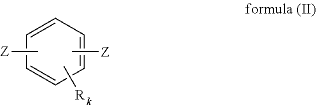

wherein
each of Z, equal to or different from each other, are selected from the group consisting of a chloride, a bromide, an iodide, an alkanesulfonate or a fluoroalkanesulfonate anion, preferably, each of Z is a chloride anion,
R and k, have the meanings given above;
   and wherein the process is carried out in the presence of a catalyst compound [compound (C), herein after] wherein said catalyst compound comprises a metal selected from the group consisting of palladium, cobalt and nickel, thereby providing a diketo compound [compound (KK), herein after] of formula (III):

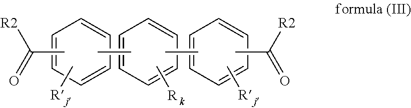

wherein R2, R′, R, k and j′, have the meanings given above;

Step 2. Bayer-Villiger oxidation of the compound (KK) of formula (III) provided in Step 1, thereby providing a diester compound [compound (EE), herein after] of formula (IV):

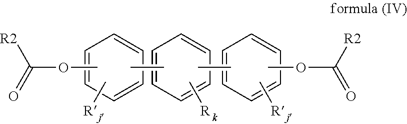

wherein R2, R′, R, k and j′, have the meanings given above;

Step 3. hydrolysis or alcoholysis of the compound (EE) of formula (IV) provided in Step 2, thereby forming compound (T) of formula (T), as detailed above.

In formula (T), the respective phenylene moieties may independently have 1,2-, 1,4- or 1,3-linkages to the other moieties different from R or R′ in the formula (T). Preferably, said phenylene moieties have 1,3- or 1,4- linkages, more preferably they have 1,4-linkage.

Still, in formula (T), j′ and k are preferably at each occurrence zero, that is to say that the phenylene moieties have no other substituents than those enabling linkage in the main chain of the polymer.

Thus, preferred compounds (T) of the present invention are selected from a group consisting of 1,1′:4′,1″-terphenyl-4,4″-diol, 1,1′:3′,1″-terphenyl-4,4″-diol, 1,1′:2′,1″-terphenyl-4,4″-diol.

Within the context of the present invention the mention “at least one organozinc compound of formula (I)” is intended to denote one or more than one organozinc compound of formula (I). Mixtures of organozinc compounds of formula (I) can advantageously be used for the purposes of the invention.

In the rest of the text, the expressions “organozinc compound of formula (I)” are understood, for the purposes of the present invention, both in the plural and the singular, that is to say that in the process of the present invention may comprise one or more than one organozinc compound of formula (I) may be reacted.

It is further understood that the zinc atom in the organozinc compound of formula (I) is directly bonded to, or coordinated with, the carbon atom of the aromatic moiety (Zn<—C) via metal coordination complex bonding.

In the organozinc compound of formula (I), R2 is preferably a C₁-C₁₀-alkyl more preferably a C₁-C₄-alkyl and even a methyl or ethyl.

Among preferred organozinc compounds of formula (I), as detailed above, suitable for being used in the process of the present invention, mention may be notably made of zinc, (4-acetylphenyl)chloro-, (3-acetylphenyl)chloro-, (2-acetylphenyl)chloro-, (4-acetylphenyl)bromo-, (4-acetylphenyl)iodo-, 4-(acetylphenyl)methanesulfonato-, 4-(acetylphenyl)trifluoromethanesulfonato-, (4-propionylphenyl)chloro-, (4-pivaloylphenyl)chloro-, (4-trifluoroacetylphenyl)chloro-. Most preferred organozinc compound of formula (I) is zinc, (4-acetylphenyl)chloro-.

In Step 1. of the process for the manufacture of the compound (T) of formula (T), the organozinc compounds of formula (I) can be prepared according to standard preparation methods such as notably disclosed by P. Knochel, R. D. Singer, in Chem. Rev. 1993, 93, 2117 and by C. Gosmini, Y. Rollin, J.-Y. Nédélec, J. Périchon, J. Org. Chem. 2000, 65, 6024, which are hereby incorporated herein by reference in their entirety. Preferably, the organozinc compound of formula (I) can also be prepared by a synthesis method including the use of zinc dust, a cobalt salt, a zinc salt and a polar aprotic solvent such as notably described in US 2004/0236155 and notably described by Gosmini et al, Synlett, 2006, N6, P 881-884, which are hereby incorporated herein by reference in their entirety.

Generally, said organozinc compounds of formula (I) are prepared in-situ in a separate reaction step prior to Step 1. of the process of the present invention.

Typically, the concentration of the organozinc, as formed, can be determined according to known practice in the art, in particular by using for example Gas Chromatography methods, thereby using iodine as a quenching agent.

It is further understood that when the organozinc compound of formula (I), as detailed above, have been prepared by using Zn dust, as mentioned above, then it is preferred that the excess of Zn dust present in the organozinc compound of formula (I) is removed prior to Step 1. of the process of the present invention.

The excess of Zn dust can be removed according to standard practice.

This being said, the amount of residual Zinc dust present in the organozinc compound of formula (I) is advantageously present in an amount of below 2 moles %, preferably below 1 mole % and more preferably below 0.5 mole %, relative to the total molar amount of organozinc compound of formula (I).

Within the context of the present invention the mention “at least one dihalocompound [compound (HH), herein after] of formula (II)” is intended to denote one or more than one compound (HH). Mixtures of compound (HH) can be advantageously used for the purposes of the invention.

In the rest of the text, the expressions “compound (HH)” are understood, for the purposes of the present invention, both in the plural and the singular, that is to say that in the process of the present invention one or more than one compound (HH) may be reacted.

Among preferred compound (HH), as detailed above, suitable for being used in Step 1. of the process for the manufacture of the compound (T) of formula (T), mention may be notably made of 1,4-dichlorobenzene, 1,4-dibromobenzene, 1,4-diodobenzene, 1,3-dichlorobenzene, 1,2-dichlorbenzene, benzene-1,4-diyl dimethanesulfonate, benzene-1,4-diyl bis(trifluoromethanesulfonate). Most preferred compound (HH) is 1,4-dichlorobenzene.

In Step 1. of the process for the manufacture of the compound (T) of formula (T), the molar ratio of the organozinc compound of formula (I), as described above, to compound (HH), as described above, is advantageously equal to or above 2.0:1.0, preferably equal to or above 2.1:1.0, more preferably equal to or above 2.2:1.0 and most preferably equal to or above 2.3:1.0.

In one embodiment of the process according to the invention, the molar ratio of the organozinc compound of formula (I), as described above, to compound (HH), as described above, is advantageously equal to or below 6.0:1.0, preferably below 5.0:1.0, more preferably below 4.0:1.0 and most preferably below 3.5:1.0.

The reaction of the at least one organozinc compound of formula (I), as detailed above, with the at least one compound (HH) of formula (II), as detailed above, in the presence of compound (C), as detailed above, in Step 1. of the process of the present invention, is often referred to as a Negishi Coupling reaction.

In a preferred embodiment, the compound (C), as detailed above, suitable for being used in Step 1. of the process for the manufacture of the compound (T) of formula (T), may be selected from a group of compounds including, but not being limited to, palladium catalysts, cobalt catalysts, nickel catalysts and mixtures thereof.

A large number of suitable palladium catalysts, generally used in aryl-aryl Negishi Coupling reactions, such as notably disclosed in in Chem. Rev. 1993, 93, 2173-2178, as mentioned above, can advantageously be used, in Step 1. of the process of the present invention.

Examples of suitable palladium catalyst include, but are not limited to, tetrakis(triphenylphosphine)palladium(0) [Pd(Ph₃P)₄]; tris(dibenzylideneacetone)dipalladium(0) [Pd₂(dba)₃]; palladium(II) acetate [Pd(OAc)₂], palladium chloride [PdCl₂], bis(benzonitrilc)dichloropalladium [Pd(PhCN)₂C1₂], palladium dichloride dichloromethane complex [Pd(dppf)₂C1₂ CH₂C1₂], PdCl₂.2TPP, bis(triphenylphosphine) palladium (II) chloride, bis(tricyclohexylphosphine) palladium (ii) chloride, bis(triphenylphosphine) palladium (II) acetate, and mixture thereof. Preferably, the palladium catalyst is PdCl₂.2TPP.

According to a particular embodiment, the palladium catalyst, as described above, can suitably be present in the form of a complex with at least one ligand (Ip_{d}) such as notably Ph₃P, and/or used in the presence of at least one ligand (Hᵢₘ), being equal of different from ligand

The at least one ligand (Hᵢₘ) is advantageously added separately to the reaction mixture.

Examples of suitable ligands (Hᵢₘ) include, but are not limited to, biarylphosphine ligands such as notably disclosed by J. E. Milne and S. L. Buchwald in J. Am. Chem. Soc. 2004, 126, 13028-13032 and by C. Han and S. L. Buchwald in J. Am. Chem. Soc. 2009, 131, 7532-7533, which are hereby incorporated herein by reference in its entirety.

The molar ratio of ligand (Ip_{d}) and/or ligand (Hᵢₘ) to palladium catalyst is typically in the range from 2:1 to 15:1.

Examples of suitable cobalt catalysts include, but are not limited to, cobalt bromide, cobalt chloride.

Preferably, the cobalt catalyst is cobalt bromide.

According to a particular embodiment, the cobalt catalyst can be used in the presence of at least one ligand wherein said ligand is advantageously added separately to the reaction mixture.

Examples of suitable ligands include, but are not limited to, triphenylphosphine, bipyridine, triphenylphosphite, tricyclohexylphosphine.

The molar ratio of said ligand to the cobalt catalyst is typically in the range from 2:1 to 15:1.

A large number of suitable nickel catalysts, generally used in aryl-aryl Negishi Coupling reactions, such as notably disclosed by B.M. Rosen et al. in Chem. Rev., 2011, 111 (3), pp 1371-1399-1402, which is hereby incorporated herein by reference in its entirety, can advantageously be used, in Step 1. of the process of the present invention.

Examples of suitable nickel catalyst include, but are not limited to, tetrakis(triphenylphosphine)Ni(0) [Ni(Ph₃P)₄], bis(triphenylphosphine) nickel (II) chloride [NiCl₂.2P(OPh)₃], bis(triphenylphosphite) nickel(II) chloride, bis(tricyclohexylpphosphine) nickel(II) chloride [NiCl₂.2P(cyclohexyl)₃], nickel acetate [Ni(OAc)₂], NiCl₂(dppe), NiCl₂(dppf), nickel chloride [NiCl₂].

According to a particular embodiment, the nickel catalyst, as described above, can suitably be present in the form of a complex with at least one ligand (I_{Ni}) such as notably Ph₃P, and/or used in the presence of at least one ligand (II_{Ni}), being equal of different from ligand (I_{Ni}).

The at least one ligand (II_{Ni}) is advantageously added separately to the reaction mixture.

Examples of suitable ligands (I_{Ni}) and (II_{Ni}), include, but are not limited to, triphenylphosphine, bipyridine, triphenylphosphite, tricyclohexylphosphine.

According to a particular preferred embodiment, the nickel catalyst is NiCl₂.2TPP and said NiCl₂.2TPP is present in complex with Ph₃P.

The molar ratio of ligand (I_{Ni}) and/or ligand (II_{Ni}) to nickel catalyst is typically in the range from 2:1 to 15:1.

If desired, compound (C), as detailed above, in Step 1. is used in combination with Zinc dust.

The molar ratio of said Zinc dust to compound (C) is advantageously equal to or above 0.95:1.00, preferably equal to or above 0.97:1.00, more preferably equal to or above 1.00:1.00. On the other hand, the molar ratio of said Zinc dust to compound (C) is advantageously equal to or below 2.00:1.00, preferably equal to or below 1.80:1.00, more preferably equal to or above 1.50:1.00 and even more preferably equal to or above 1.25:1.00.

In Step 1. of the process according to the invention, the ratio of the total molar amount of compound (C) to the molar amount of compound (HH) is generally equal to or above 0.001, preferably equal to or above 0.005, preferably equal to or above 0.008.

In Step 1. of the process according to the invention, the ratio of the total molar amount of compound (C) to the molar amount of compound (HH) is generally equal to or below 0.1, preferably equal to or 0.08, preferably equal to or below 0.06.

Very good results have been obtained with ratio of the total molar amount of compound (C) to the molar amount of compound (HH) of equal to or above 0.008 and equal to or below 0.06.

In Step 1. of the process for the manufacture of the compound (T) of formula (T), the reaction is generally carried out in the presence of a solvent.

Suitable solvents for use in Step 1. of the process include, not limited to, N-methylpyrrolidone (NMP), N-ethylpyrrolidone, N,N-dimethylformamide DMF, N,N-dimethylacetamide DMAc, tetrahydrofuran THF, dioxane and mixtures thereof.

Step 1. of the process according to the present invention is preferably carried out at a temperature T1 of below 120° C., more preferably of below 110° C., still more preferably of below 100° C. and most preferably of below 90° C. On the other hand, the process according to the present invention is preferably carried out at a temperature T1 of above 5° C., more preferably of above 10° C., still more preferably of above 20° C. and most preferably of above 30° C.

Step 1. of the process according to the present invention is preferably carried out during a reaction time t₁ of below 16 hours, more preferably of below 12 hours, still more preferably of below 10 hours and most preferably of below 9 hours. On the other hand, the process according to the present invention is preferably carried out during a reaction time t₁ of above 0.5 hour, more preferably of above 1 hours, still more preferably of above 2 hours and most preferably of above 3 hours.

In Step 1. of the process according to the present invention, the reactor is advantageously used while taking care to avoid the presence of any reactive gases in the reactor. These reactive gases may be notably oxygen, water and carbon dioxide. O₂ and water are the most reactive and should therefore be avoided.

In a particular embodiment in Step 1. of the process according to the present invention, the reactor should be evacuated under pressure or under vacuum and filled with an inert gas containing less than 20 ppm of reactive gases, and in particular less than 10 ppm of O₂ and less than 10 ppm water prior to adding the reactants to the reaction mixture. Then, the reactor should be put under a constant purge of said inert gas until the end of the reaction. The inert gas is any gas that is not reactive under normal circumstances. It may be chosen from nitrogen, argon or helium. The inert gas contains preferably less than 10 ppm oxygen, 10 ppm water and 20 ppm carbon dioxide.

According to certain embodiments, Step 1. of the process according to the present invention is preferably carried out at a pressure of below 10 atm, more preferably of below 7 atm, still more preferably of below 5 atm and most preferably of below 2 atm. On the other hand, Step 1. of the process according to the present invention is preferably carried out at a temperature of above 0.5 atm, more preferably of above 0.6 atm, still more preferably of above 0.7 atm and most preferably of above 0.8 atm. Excellent results were obtained when Step 1. of the process according to the present invention was carried out at atmospheric pressure.

According to one embodiment, in Step 1. of the process of the present invention, the compound (HH), as detailed above, the compound (C), as detailed above, optionally, the ligands, as detailed above, optionally the Zinc dust, optionally the solvent and the organozinc compound, as detailed above, are added to the reactor at the same time.

According to a preferred embodiment, in Step 1. of the process of the present invention, the compound (HH), as detailed above, the compound (C), as detailed above, optionally, the ligands, as detailed above, optionally the Zinc dust, and optionally the solvent are first added to the reactor and the organozinc compound, as detailed above, is then generally added slowly to said reaction mixture.

If desired, Step 1. of the process according to the present invention can be quenched by adding in the end of Step 1. a quenching compound selected from the group consisting of aqueous inorganic acid, an organic acid, an alkanol, and a halo-ano line compound.

Preferred quenching compounds can be chosen among hydrochloric acid, sulfuric acid, acetic acid, methanol, 4-chloroaniline, 3-chloroaniline and 2-chloroaniline.

In compound (KK) of formula (III), the respective phenylene moieties may independently have 1,2-, 1,4- or 1,3-linkages to the other moieties different from R or R′ in the formula (KK). Preferably, said phenylene moieties have 1,3- or 1,4-linkages, more preferably they have 1,4-linkage.

Still, in formula (KK) j′ and k are preferably at each occurrence zero, that is to say that the phenylene moieties have no other substituents than those enabling linkage in the main chain of the polymer.

In compound (KK) of formula (III), R2 is preferably a C₁-C₁₀-alkyl more preferably a C₁-C₄-alkyl and even a methyl or ethyl.

Thus, preferred compounds (KK) of the present invention are selected from a group consisting of 1,1′:4′,1″-terphenyl-4,4″-diacetyl, 1,1′:3′,1″-terphenyl-4,4″-diacetyl, 1,1′:2′,11″-terphenyl-4,4″-diacetyl.

In Step 1. of the process according to the present invention, the compound (KK) may be isolated from the reaction medium by precipitation or liquid-liquid extraction.

Good results were obtained when the compound (KK) of formula (III) and in particular 1,1′:4′,1″-terphenyl-4,4″-diacetyl was isolated by precipitation by adding a non solvent thereby providing an isolated compound (KK) of formula (III), in particular isolated 1,1′:4′,1″-terphenyl-4,4″-diacetyl.

Suitable non solvents may include, but not limited to, water, methanol, ethanol, and acetone.

According to another embodiment in Step 1. of the process, the compound (KK) of formula (III) and in particular 1,1′:4′,1″-terphenyl-4,4″-diacetyl was isolated by liquid-liquid extraction. Said liquid-liquid extraction can typically be carried out by the addition of water and an organic water immiscible solvent such as notably methyl isobutyl ketone (MIBK), toluene, xylene, 1,2,4-trimethylbenzene, 1,2,3-trimethylbenzene, 1,2-dichlorobenzene, 1,3-dichlorobenzene, 1,2,4-trichlorobenzene, and the like.

According to certain embodiments, the isolated compound (KK) of formula (III), in particular isolated 1,1′:4′,1″-terphenyl-4,4″-diacetyl is further purified by standard purification methods such as notably by crystallization, extraction, such as liquid-liquid extraction, distillation under vacuum thereby providing a purified and isolated compound (KK) of formula (III), in particular purified and isolated 1,1′:4′,1″-terphenyl-4,4″-diacetyl.

Said purified and isolated compound (KK) of formula (III), in particular purified and isolated 1,1′:4′,1″-terphenyl-4,4″-diacetyl can then be submitted to the Bayer-Villiger oxidation in Step 2. of the process according to the present invention.

According to preferred embodiments, the isolated compound (KK) of formula (III), in particular isolated 1,1′:4′,1″-terphenyl-4,4″-diacetyl, is directly, thus without any additional purification, submitted to the Bayer-Villiger oxidation in Step 2. of the process according to the present invention.

In Step 2. of the process according to the present invention, the Bayer-Villiger oxidation is carried out using at least one Bayer-Villiger oxidant.

For the purpose of the present invention, the term “Bayer-Villiger oxidants” is intended to denote all oxidants which are known to the person skilled in the art for Bayer-Villiger oxidations.

The Bayer-Villiger oxidants can be employed either in pure form or in the form of their mixtures.

According to certain embodiments in Step 2. of the process according to the present invention, the Bayer-Villiger oxidant is chosen from the group consisting of inorganic or organic peroxides, hydrogen peroxide, an adduct of hydrogen peroxide and urea, peroxo complexes of transition metals, organic peracids, inorganic peracids, dioxiranes or mixtures thereof.

It is further understood that the organic or inorganic peracids may be formed in situ in the reaction mixture from a mixture of hydrogen peroxide with an organic acid and/or an inorganic acid, respectively.

Among preferred inorganic peroxides, suitable for being used in Step 2. of the process according to the present invention, mention may be notably made of an ammonium peroxide, an alkali metal peroxide, an ammonium persulfate, an alkali metal persulfate, an ammonium perborate, an alkali metal perborate, an ammonium percarbonate, an alkali metal percarbonate, an alkaline-earth metal peroxide, zinc peroxide or a mixture of these oxidants.

Among preferred organic peroxides, suitable for being used in Step 2. of the process according to the present invention, mention may be notably made of tert-butyl hydroperoxide, cumene hydroperoxide, menthyl hydroperoxide, 1-methylcyclohexane hydroperoxide or a mixture thereof.

Among preferred peroxo complexes of transition metals, suitable for being used in Step 2. of the process according to the present invention, mention may be notably made of peroxo complexes of the transition metals iron, manganese, vanadium or molybdenum or mixtures of these peroxo complexes. It is further understood that the peroxo complex may contain two or more transition metals, being equal or different from each other.

Among preferred organic peracids, suitable for being used in Step 2. of the process according to the present invention, mention may be notably made of perbenzoic acid, m-chloroperbenzoic acid, magnesium monoperphthalic acid, peracetic acid, performic acid, peroxytrifluoroacetic acid or a mixture thereof.

Among preferred inorganic peracids, suitable for being used in Step 2. of the process according to the present invention, mention may be notably made of peroxymonosulfuric acid (also called Caro's acid), peroxyphosphoric acid (H₃PO₅).

Among preferred organic peracids formed in situ from a mixture of hydrogen peroxide with an organic acid, suitable for being used in Step 2. of the process according to the present invention, mention may be notably made of peracetic acid formed in situ from hydrogen peroxide, sulfuric acid and acetic acid.

Among preferred inorganic peracids formed in situ from a mixture of hydrogen peroxide with an inorganic acid, suitable for being used in Step 2. of the process according to the present invention, mention may be notably made of an inorganic peracids formed in situ from hydrogen peroxide with boron trifluoride.

Good results have been obtained by using peracetic acid as oxidant in Step 2. of the process according to the present invention which was formed in situ from hydrogen peroxide, sulfuric acid and acetic acid.

In Step 2. of the process according to the invention, the ratio of the total molar amount of compound (KK), as detailed above, to the molar amount of the Bayer-Villiger oxidant is generally equal to or above 0.01, preferably equal to or above 0.05, preferably equal to or above 0.10, preferably equal to or above 0.15.

In Step 2. of the process according to the invention, the ratio of the total molar amount of compound (KK), as detailed above, to the molar amount of the Bayer-Villiger oxidant is generally equal to or below 0.75, preferably equal to or below 0.50, preferably equal to or below 0.45, preferably equal to or below 0.40.

Very good results have been obtained with ratio of the total molar amount of compound (KK) to the molar amount of the Bayer-Villiger oxidant of equal to or above 0.05 and equal to or below 0.50.

In Step 2. of the process for the manufacture of the compound (T) of formula (T), the reaction is generally carried out in the presence of a solvent, such as notably toluene, xylene, 1,2,4-trimethylbenzene, 1,2,3-trimethylbenzene, 1,2-dichlorobenzene, 1,3-dichlorobenzene, 1,2,4-trichlorobenzene or additional acid, such as notably acetic acid and trifluoroacetic acid.

Step 2. of the process according to the present invention is preferably carried out at a temperature T2 of below 70° C., more preferably of below 65° C., still more preferably of below 60° C. and most preferably of below 55° C. On the other hand, the process according to the present invention is preferably carried out at a temperature T2 of above 5° C., more preferably of above 15° C., still more preferably of above 25° C. and most preferably of above 30° C.

Step 2. of the process according to the present invention is preferably carried out during a reaction time t₂ of below 20 hours, more preferably of below 18 hours, still more preferably of below 16 hours and most preferably of below 14 hours. On the other hand, the process according to the present invention is preferably carried out during a reaction time t₂ of above 1 hour, more preferably of above 8 hours, still more preferably of above 9 hours and most preferably of above 10 hours. Good results were obtained when Step 1. was carried out during a reaction time t₂ of 12 hours.

In Step 2. of the process according to the present invention, prior to adding the compound (KK), as detailed above, the Bayer Villiger oxidant, as detailed above, and optionally, the solvent and/or additional acid to the reactor, said reactor is advantageously pursued while taking care to avoid the presence of too high amounts of oxygen to assure nonflammable conditions in the reactor.

In a particular embodiment in Step 2. of the process according to the present invention, the reactor should be evacuated under pressure or under vacuum and filled with an inert gas containing less than 1000 ppm of O₂, preferably less than 100 ppm of O₂ prior to adding the reactants to the reaction mixture. Then, the reactor should be put under a constant purge of said inert gas until the end of the reaction. The inert gas is any gas that is not reactive under normal circumstances. It may be chosen from nitrogen, argon or helium.

According to certain embodiments, Step 2. of the process according to the present invention is preferably carried out at a pressure of below 10 atm, more preferably of below 7 atm, still more preferably of below 5 atm and most preferably of below 2 atm. On the other hand, Step 2. of the process according to the present invention is preferably carried out at a temperature of above 0.5 atm, more preferably of above 0.6 atm, still more preferably of above 0.7 atm and most preferably of above 0.8 atm. Excellent results were obtained when Step 2. of the process according to the present invention was carried out at atmospheric pressure.

In compound (EE) of formula (IV), the respective phenylene moieties may independently have 1,2-, 1,4- or 1,3-linkages to the other moieties different from R or R′ in the formula (IV). Preferably, said phenylene moieties have 1,3- or 1,4-linkages, more preferably they have 1,4-linkage.

Still, in compound (EE) of formula (IV), j′ and k are preferably at each occurrence zero, that is to say that the phenylene moieties have no other substituents than those enabling linkage in the main chain of the polymer.

In formula (EE) of formula (IV), R2 is preferably a C₁-C₁₀-alkyl more preferably a C₁-C₄-alkyl and even more preferably, a methyl or ethyl.

Thus, preferred compounds (EE) of formula (IV) of the present invention are selected from a group consisting of 1,1′:4′,1″-terphenyl-4,4″-diacetate, 1,1′:3′,1″-terphenyl-4,4″-diacetate, 1,1′:2′,11″-terphenyl-4,4″-diacetate.

In Step 2. of the process according to the present invention, the compound (EE) of formula (IV), may be isolated from the reaction medium by precipitation or liquid-liquid extraction thereby providing an isolated compound (EE) of formula (IV), in particular isolated 1,1′:4′,1″-terphenyl-4,4″-diacetyl.

Good results were obtained when the compound (EE) of formula (IV) and in particular 1,1′:4′,1″-terphenyl-4,4″-diacetate was isolated by precipitation by adding a non solvent.

Suitable non solvents may include, but not limited to, water, methanol, ethanol, and acetone.

According to one embodiment of the present invention, at the end of Step 2, the reaction medium prior to isolation of the isolated compound (EE) of formula (IV), in particular isolated 1,1′:4′,1″-terphenyl-4,4″-diacetate, is treated with a reducing agent.

The reducing agent may be present in the form of a solid or in the form of an aqueous or organic solution.

According to a preferred embodiment, the reducing agent is present in the form of an aqueous solution.

According to another preferred embodiment, the reducing agent is present in the form of an organic solution, in particular an alcoholic solution.

Suitable reducing agents may include, but not limited to sodium sulfite, potassium sulfite, lithium sulfite, sodium hydrogen sulfite, potassium hydrogen sulfite, manganese dioxide and mixtures thereof.

According to another preferred embodiment of the present invention, the isolated compound (EE) of formula (IV), in particular isolated 1,1′:4′,1″-terphenyl-4,4″-diacetate, is treated with a reducing agent, as detailed above.

According to a specific preferred embodiment, the isolated compound (EE) of formula (IV), in particular isolated 1,1′:4′,1″-terphenyl-4,4″-diacetate, is added to a solution comprising the reducing agent in an amount of equal to or at least 5% by weight (wt %), preferably equal to or at least 10 wt %.

The Applicant has found that said reducing agent advantageously decomposes the excess of the Bayer-Villier oxidant, as detailed above, which is still present in the reaction medium or in the isolated compound (EE) of formula (IV), in particular isolated 1,1′:4′,1″-terphenyl-4,4″-diacetate.

According to another embodiment in Step 2. of the process, the compound (EE) of formula (IV) and in particular 1,1′:4′,1″-terphenyl-4,4″-diacetate was isolated by liquid-liquid extraction. Said liquid-liquid extraction can typically be carried out by the addition of water and an organic water immiscible solvent such as notably methyl isobutyl ketone (MIBK), toluene, xylene, 1,2,4-trimethylbenzene, 1,2,3-trimethylbenzene, 1,2-dichlorobenzene, 1,3-dichlorobenzene, 1,2,4-trichlorobenzene, and the like.

According to certain embodiments, the isolated compound (EE) of formula (IV), in particular isolated 1,1′:4′,1″-terphenyl-4,4″-diacetate is further purified by standard purification methods such as notably by crystallization, extraction, such as liquid-liquid extraction, distillation under vacuum thereby providing a purified and isolated compound (EE) of formula (IV), in particular purified and isolated 1,1′:4′,1″-terphenyl-4,4″-diacetate.

Said purified and isolated compound (EE) of formula (IV), in particular purified and isolated 1,1′:4′,1″-terphenyl-4,4″-diacetate can then be further hydrolyzed or alcoholised in Step 3. of the process according to the present invention.

According to preferred embodiments, the isolated compound (EE) of formula (IV), in particular isolated 1,1′:4′,1″-terphenyl-4,4″-diacetate, is directly submitted to a hydrolysis or alcoholysis in Step 3. of the process according to the present invention.

In Step 3. of the process according to the present invention, the hydrolysis or alcoholysis can be carried out under acid catalysis or basic catalysis.

According to one embodiment, Step 3. of the process according to the present invention, the hydrolysis is carried out under acid catalysis wherein compound (EE) of formula (IV), in particular 1,1′:4′,1″-terphenyl-4,4″-diacetate, as isolated, optionally purified from Step 2, is generally contacted with an acid aqueous or an acid aqueous alcoholic solution (e.g. an aqueous or an aqueous alcoholic solution of HCl, H₂SO₄, CH₃COOH).

According to another embodiment, Step 3. of the process according to the present invention, the hydrolysis is carried out under basic catalysis wherein compound (EE) of formula (IV), in particular 1,1′:4′,1″-terphenyl-4,4″-diacetate, as isolated, optionally purified from Step 2, is generally contacted with a base in an aqueous or an alcoholic solution or an aqueous alcoholic solution.

According to a preferred embodiment, Step 3. of the process according to the present invention, the alcoholysis is carried out under basic catalysis wherein compound (EE) of formula (IV), in particular 1,1′:4′,1″-terphenyl-4,4″-diacetate, as isolated, optionally purified from Step 2, is generally contacted with a base in an alcoholic solution.

Among suitable bases mention can be made of, but not limiting to, sodium hydroxide, potassium hydroxide, lithium hydroxide, sodium methano late, potassium methano late, sodium t-butanolate, potassium t-butanolate. Sodium and potassium hydroxide are preferred.

In general, in Step 3, after adding said acid aqueous or an acid aqueous alcoholic solution, or a base in an aqueous, alcoholic or aqueous alcoholic solution, and the isolated, optionally purified compound (EE) of formula (IV), in particular isolated, optionally purified 1,1′:4′,1″-terphenyl-4,4″-diacetate, the temperature is raised to a temperature T3 and preferably maintained in Step 3. to a temperature T3.

The temperature T3 in Step 3. is preferably of below 120° C., more preferably of below 110° C., still more preferably of below 100° C. and most preferably of below 90° C. On the other hand, the temperature T3 is preferably of above 40° C., more preferably of above 50° C., still more preferably of above 60° C.

Step 3. of the process according to the present invention is preferably carried out during a reaction time t₃ of below 20 hours, more preferably of below 18 hours, still more preferably of below 16 hours and most preferably of below 14 hours. On the other hand, the process according to the present invention is preferably carried out during a reaction time t₃ of above 1 hour, more preferably of above 6 hours, still more preferably of above 7 hours and most preferably of above 8 hours. Good results were obtained when Step 1. was carried out during a reaction time t₃ of 10 hours.

In Step3, the compound (T) may be isolated from the reaction medium by precipitation, crystallization or extraction, which can be carried out according to standard practice of the skilled in the art.

Good results were obtained when the compound (T) and in particular 1,1′:4′,1″-terphenyl-4,4″-diol was isolated by precipitation by adding for example water, by liquid-liquid extraction or by distillation under vacuum.

According to one embodiment, the process of the present invention is carried out in one pot. The term “one pot” when referred to a reaction is generally intended to denote any reaction where a reactant is subjected to successive chemical reactions in just one reactor, thereby avoiding a lengthy separation process and purification of the intermediate chemical compounds.

Thus, all Steps 1. to 3. may all be carried out in one reactor.

According to certain embodiment, the process of the present invention is carried out in at least two or more pots, preferably in three pots.

According to a preferred embodiment, each of Steps 1. to 3 of the process of the present invention is carried out in a separate reactor.

Should the disclosure of any patents, patent applications, and publications which are incorporated herein by reference conflict with the description of the present application to the extent that it may render a term unclear, the present description shall take precedence.

The invention will be now described in more details with reference to the following examples, whose purpose is merely illustrative and not intended to limit the scope of the invention.

### Raw Materials

N-methylpyrrolidinone HPLC grade, procured from Aldrich and dried on MS4A to <50 ppm water

Acetonitrile, HPLC grade, procured from Aldrich and dried on MS4A to <50 ppm water

Zinc dust, 325 mesh, was procured from Aldrich.

1 M anhydrous solution of HCl in diethyl ether was procured from Aldrich

Potassium hydroxide, bis(triphenylphosphine) nickel(II) chloride, bis(triphenylphosphine) palladium(II) chloride, triphenylphosphine, cobalt(II) bromide, zinc bromide, pyridine, 4-chloroacetophenone, 1,4-dichlorobenzene, 4-chloroaniline were procured from Aldrich and used as received. Manufacturing of 1,1′:4;1″-terphenyl-4,4″-diol

### COMPARATIVE EXAMPLE 1

### Prepared According to Example 2 from EP 0 343 798 A1

In a 1000 mL 4-neck glass reactor fitted with a stirrer, a N₂ inlet tube, a Claisen adapter with a thermocouple plunging in the reaction medium, and a Barrett trap with a condenser were introduced 500.00 g of phenol (5.32 mol), 19.64 g of concentrated HCl (0.199 mol). Using a powder dispenser, 37.261 g of 1,4-cyclohexanedione (0.332 mol) were added to the mixture over a period of 2 hours. The mixture was held at 50° C. for 7 hours. Providing cooling with an ice batch, 24.95 g of sodium hydroxide, and 1.663 g of palladium on carbon were added slowly to the mixture. Under nitrogen the reaction mixture was then heated to 180° C. and held at that temperature for 4 hours during which period, 15.53 g of distillate were collected. At the end of the reaction 50 g of glacial acetic acid and 85 g of water were added, causing a solid to precipitate. The solid (4.38 g) was isolated by filtration on Büchner funnel, dried at 120° C. at 50 mbar and analyzed by HPLC (1,1′:4′,1″-terphenyl-4,4″-diol, 49% purity, 2.5% yield). The two liquid phases (filtrate) were separated in a separation funnel and the organic phase (1309.99 g) was analyzed by HLPC to contain 0.13% of 1,1′:4′,1″-terphenyl-4,4″-diol (2.1% yield).

The total yield is thus 4.6% yield.

### EXAMPLE 2

### Preparation of Activated Zinc Dust

In a 1000 mL 4-neck glass reactor fitted with a stirrer, a N₂ inlet tube, a Claisen adapter with a thermocouple plunging in the reaction medium, and a condenser were introduced 100 g of commercially available zinc dust and 500 mL of 1 M hydrogen chloride in diethyl ether (anhydrous). The mixture was slurried for 2 hours then the supernatant was removed by filtration. The washing of the zinc dust was repeated one more time with 500 g of HCl in diethyl ether followed by 2 washings with anhydrous diethyl ether. The solid was then dried in vacuo or under an inert atmosphere for several hours at 100-200° C. The final zinc dust was isolated in a glove box, by sieving on 150 mesh sieve. The activated zinc dust should be used immediately or stored under an inert atmosphere away from oxygen and moisture.

### EXAMPLE 3

### Preparation of zinc, (4-acetylphenyl)chloro—(i.e. the organozinc Compound of Formula (I))

In a 2000 mL 4-neck glass reactor fitted with a stirrer, a N₂ inlet tube, a Claisen adapter with a thermocouple plunging in the reaction medium, and a Barrett trap with a condenser were introduced under an inert atmosphere, 500 mL of acetonitrile, 92.75 g 4-chloroacetophenone (0.600 mol), of CoBr₂ (0.060 mol), 54.05 g ZnBr₂ (0.240 mol), 78.47 g of activated zinc dust (1.200 mol) and 330 mL of pyridine. The reaction mixture was stirred at room temperature for 4 hours then 250 mL of NMP were added and the mixture was heated to 85° C. to distill off the acetonitrile thereby providing a supernatant liquid liquid containing less than 5wt % acetonitrile.

### EXAMPLE 4

### Step 1. by Using NiCl₂.2TPP as Compound (C)

In a 2000 mL 4-neck glass reactor fitted with a stirrer, a N₂ inlet tube, a Claisen adapter with a thermocouple plunging in the reaction medium, and a condenser were introduced under an inert atmosphere, 350 mL of NMP, 29.40 g 1,4-dichlorobenzene (0.200 mol), 0.523 g of NiCl₂.2TPP (0.0008 mol) and 1.050 g triphenylphosphine (0.004 mol), and 0.10 g of activated zinc dust (0.0015 mol) at room temperature. The supernatant liquid as recovered from example 3 was then added to the reaction at room temperature. After the addition, the reaction mixture was was heated to 80° C. and kept at 80° C. for 8 h. At the end of the reaction, the reaction was quenched by addition of 40 g of conc. hydrochloric acid, then 300 mL water. A solid had formed in the reaction mixture over the course of the reaction and was isolated by filtration. The solid (112.10 g) was isolated by filtration on Buchner funnel and rinsed with 400 mL methanol then dried at 120° C. at 50 mbar and analyzed by HPLC (1,1′:4′,1″-terphenyl-4,4″-diacetyl, 39% purity, 70% yield). The crude 1,1′:4′,1″-terphenyl-4,4″-diacetyl product was used without purification in example 7, see below.

### EXAMPLE 5

### Step 1. by Using NiCl₂.2TPP, as Compound (C) but Additional Quenching with of 4-chloroaniline

In a 2000 mL 4-neck glass reactor fitted with a stirrer, a N₂ inlet tube, a Claisen adapter with a thermocouple plunging in the reaction medium, and a condenser were introduced under an inert atmosphere, 350 mL of NMP, 29.40 g 1,4-dichlorobenzene (0.200 mol), 0.523 g of NiCl₂.2TPP (0.0008 mol) and 1.050 g triphenylphosphine (0.004 mol), and 0.10 g of activated zinc dust (0.0015 mol) at room temperature. The supernatant liquid recovered from example 3 was then added to the reaction at room temperature. After the addition, the reaction mixture was heated to 80° C. and kept at 80° C. for 8 h. At the end of the reaction, the reaction was quenched by addition of 15.31 g of 4-chloroaniline (0.120 mol) and left under agitation for another 2 hours. 40 g of conc HCl were then added to the mixture with 300 mL water. A solid had formed in the reaction mixture over the course of the reaction and was isolated by filtration. The solid (76.82 g) was isolated by filtration on Büchner funnel and rinsed with 400 mL water then dried at 120° C. at 50 mbar and analyzed by HPLC (1,1′:4′,1″-terphenyl-4,4″-diacetyl, 45% purity, 65% yield). The crude 1,1′:4′,1″-terphenyl-4,4″-diacetyl product was used without purification in example 8, see below.

### EXAMPLE 6

### Step 1. by Using PdCl₂.2TPP, as compound (C)

In a 2000 mL 4-neck glass reactor fitted with a stirrer, a N₂ inlet tube, a Claisen adapter with a thermocouple plunging in the reaction medium, and a condenser were introduced under an inert atmosphere, 350 mL of NMP, 29.40 g 1,4-dichlorobenzene (0.200 mol), 0.561 g of PdCl₂.2TPP (0.0008 mol) and 1.050 g triphenylphosphine (0.004 mol), and 0.10 g of activated zinc dust (0.0015 mol) at room temperature. The supernatant liquid recovered from example 3 was then added to the reaction at room temperature. After the addition, the reaction mixture was heated to 80° C. and kept at 80° C. for 8 h. At the end of the reaction, the reaction was quenched by addition of 40 g of conc. hydrochloric acid, then 300 mL water. A solid had formed in the reaction mixture over the course of the reaction and was isolated by filtration. The solid (105.06 g) was isolated by filtration on Buchner funnel and rinsed with 400 mL methanol then dried at 120° C. at 50 mbar and analyzed by HPLC (1,1′:4′,1″-terphenyl-4,4″-diacetyl, 37% purity, 75% yield The crude 1,1′:4′,1″-terphenyl-4,4″-diacetyl product was used without purification in example 9, see below.

### EXAMPLE 7

### Bayer-Villiger Oxidation and Hydrolysis Under Basic Catalysis (i.e. Step 2. and Step 3.)

In a 1000 mL 4-neck glass reactor fitted with a glass stirrer, a N₂ inlet tube, a Claisen adapter with a thermocouple plunging in the reaction medium, and a condenser were introduced 112.10 g of the crude 1,1′:4′,1″-terphenyl-4,4″-diacetyl obtained in example 4 (0.140 mol), 500 g of acetic acid (8.32 mol), 0.202 g of sulfuric acid (0.0019 mol) and 104.61 g of hydrogen peroxide (39 wt %, 1.200 mol). The reactor was sealed and under nitrogen was heated up to 50° C. The slurry was agitated for 12 h, then 200 g water were added and the solid isolated by filtration on Buchner funnel. The solid was slurried in a 1000 mL beaker with 500 mL of 10 wt % sodium sulfite solution, then rinsed with water. The crude solid (1,1′:4′,1″-terphenyl-4,4″-diacetate) was then reintroduced in the reactor, along with 300 mL anhydrous ethanol with 39.27 g potassium hydroxide (0.700 mol) and the slurry was heated to 78° C. and held at this temperature for 10 h. 100 g of concentrated hydrochloric acid and 400 g water were added and the solid isolated by filtration on Buchner funnel. The solid was slurried in a 1000 mL beaker with 500 mL of water, then rinsed rinsed with water until pH<7.5. The solid (39.34 g) was dried under vacuum at 120° C. overnight and analyzed by HPLC and contained 1,1′:4′,1″-terphenyl-4,4″-diol (72% purity, 54% yield).

### EXAMPLE 8

### Bayer-Villiger Oxidation and Hydrolysis Under Basic Catalysis (i.e. Step 2. and Step 3.)

Example 8 has been prepared according to the procedure of example 7 except that the crude 1,1′:4′,1″-terphenyl-4,4″-diacetyl, as obtained in example 5 was used. The final solid (30.60 g) was dried under vacuum at 120° C. overnight and analyzed by HPLC and contained 1,1′:4′,1″-terphenyl-4,4″-diol (86% purity, 50 % yield).

### EXAMPLE 9

### Bayer-Villiger Oxidation and Hydrolysis Under Basic Catalysis (i.e. Step 2. and Step 3.)

Example 8 has been prepared according to the procedure of example 7 except that the crude 1,1′:4′,1″-terphenyl-4,4″-diacetyl, as obtained in example 6 was used. The final solid (44.59 g) was dried under vacuum at 120° C. overnight and analyzed by HPLC and contained 1,1′:4′,1″-terphenyl-4,4″-diol, 65% purity, 55% yield).

1-15. (canceled)

16. A process for manufacturing a terphenyl compound (T) of formula (T): 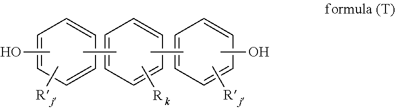
wherein
each of R and R′, equal to or different from each other, are selected from the group consisting of halogen, alkyl, aryl, ether, thioether, ester, amide, imide, alkali or alkaline earth metal sulfonate, alkyl sulfonate, alkali or alkaline earth metal phosphonate, alkyl phosphonate, amine and quaternary ammonium;
each of j′ and k, equal to or different from each other, are zero or are an integer from 1 to 4,
the process comprising:
reacting at least one organozinc compound of formula (I): 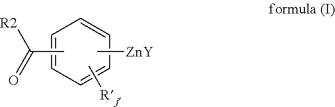
wherein
Y is selected from the group consisting of a chloride, a bromide, an iodide, an alkanesulfonate or a fluoroalkanesulfonate anion,
R2 is selected from selected from the group consisting of C₁-C₁₀-alkyl, C₃-C₁₀-cycloalkyl, C₁-C₁₀-halooalkyl, and C₃-C₁₀ halocycloalkyl,
R′ and j′, have the meanings given above,
with at least one dihalocompound (HH) of formula (II): 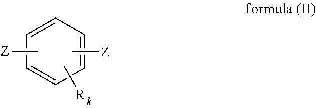
wherein
each of Z, equal to or different from each other, are selected from the group consisting of a chloride, a bromide, an iodide, an alkanesulfonate or a fluoroalkanesulfonate anion,
R and k, have the meanings given above;
and wherein the process is carried out in the presence of a catalyst compound (C) wherein said catalyst compound (C) comprises a metal selected from the group consisting of palladium, cobalt and nickel,
thereby providing a diketo compound (KK) of formula (III): 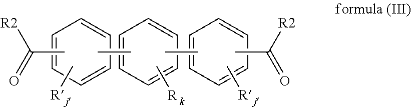
wherein R2, R′, R, k and j′, have the meanings given above;
Bayer-Villiger oxidation of the diketo compound (KK) of formula (III) providing a diester compound (EE) of formula (IV): 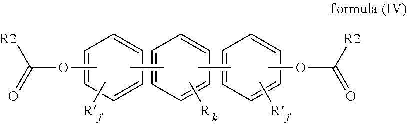
wherein R2, R′, R, k and j′, have the meanings given above;
hydrolysis or alcoholysis of the diester compound (EE) of formula (IV) forming compound (T).

17. The process according to claim 16, wherein the terphenyl compound (T) is selected from a group consisting of 1,1′:4′,1″-terphenyl-4,4″-diol and 1,1′:3′,1″-terphenyl-4,4″-diol.

18. The process according to claim 16, wherein the organozinc compound of formula (I) is selected from the group consisting of zinc, (4-acetylphenyl)chloro-, (3-acetylphenyl)chloro-, (2-acetylphenyl)chloro-, (4-acetylphenyl)bromo-, (4-acetylphenyl)iodo-, 4-(acetylphenyl)methanesulfonato-, 4-(acetylphenyl)trifluoromethanesulfonato-, (4-propionylphenyl)chloro-, (4-pivaloylphenyl)chloro-, and (4-trifluoroacetylphenyl)chloro-.

19. The process according to claim 16, wherein the dihalocompound (HH) of formula (II) is selected from the group consisting of 1,4-dichlorobenzene, 1,4-dibromobenzene, 1,4-diodobenzene, 1,3-dichlorobenzene, 1,2-dichlorbenzene, benzene-1,4-diyldimethanesulfonate, benzene-1,4-diyl bis(trifluoromethanesulfonate).

20. The process according to claim 16, wherein the molar ratio of the organozinc compound of formula (I) to the dihalocompound (HH) of formula (II) is equal to or above 2.0:1.0 and equal to or below 6.0:1.0.

21. The process according to claim 16, wherein the catalyst compound (C) is a palladium catalyst selected from the group consisting of tetrakis(triphenylphosphine)palladium(0) [Pd(Ph₃P)₄]; tris(dibenzylideneacetone)dipalladium(0) [Pd₂(dba)₃]; palladium(II) acetate [Pd(OAc)₂], palladium chloride [PdCl₂], bis(benzonitrile)dichloropalladium [Pd(PhCN)₂Cl₂], palladium dichloride dichicromethane complex [Pd(dppf)₂Cl₂ CH₂Cl₂], PdCl₂.2TPP, bis(triphenylphosphine) palladium (II) chloride, bis(tricyclohexylphosphine) palladium (ii) chloride, bis(triphenylphosphine) palladium (II) acetate, a cobalt catalyst selected from the group consisting of cobalt bromide and cobalt chloride, a nickel catalyst selected from the group consisting of tetrakis(triphenylphosphine)Ni(0) [Ni(Ph₃P)₄], bis(triphenylphosphine) nickel (II) chloride [NiCl₂.2P(OPh)₃], bis(triphenylphosphite) nickel(II) chloride, bis(tricyclohexylpphosphine) nickel(II) chloride [NiCl₂.2P(cyclohexyl)₃], nickel acetate [Ni(OAc)₂], NiCl₂(dppe), NiCl₂(dppf), and nickel chloride [NiCl₂], and mixtures thereof.

22. The process according to claim 16, wherein ratio of the total molar amount of catalyst compound (C) to the molar amount of dihalocompound (HH) is generally equal to or above 0.001 and equal to or below 0.1.

23. The process according to claim 16, wherein reacting the at least one organozinc compound with the at least one dihalocompound (HH) is carried out at a temperature T1 of below 120° C. and above 5° C., and during a reaction time t₁ of below 16 hours and above 0.5 hour.

24. The process according to claim 16, wherein the diketo compound (KK) of formula (III) is selected from a group consisting of 1,1′:4′,1″-terphenyl-4,4″-diacetyl, 1,1′:3′,1″-terphenyl-4,4″-diacetyl, and 1,1′:2′,11″-terphenyl-4,4″-diacetyl.

25. The process according to claim 16, wherein the Bayer-Villiger oxidation is carried out in the presence of at least one Bayer-Villiger oxidant and wherein said Bayer-Villiger oxidant is selected from the group consisting of inorganic or organic peroxides, hydrogen peroxide, an adduct of hydrogen peroxide and urea, peroxo complexes of transition metals, organic peracids, inorganic peracids, dioxiranes, and mixtures thereof.

26. The process according to claim 16, wherein the ratio of the total molar amount of the diketo compound (KK), to the molar amount of the Bayer-Villiger oxidant is equal to or above 0.01 and equal to or below 0.75.

27. The process according to claim 16, wherein the Bayer-Villiger oxidation is carried out at a temperature T2 of below 70° C. and above 5° C., and during a reaction t₂ of below 20 hours and above 1 hour.

28. The process according to claim 16, wherein the diester compound (EE) of formula (IV) is selected from the group consisting of 1,1′:4′,1″-terphenyl-4,4″-diacetate, 1,1′:3,1″-terphenyl-4,4″-diacetate, and 1,1′:2′,1″-terphenyl-4,4″-diacetate.

29. The process according to claim 16, wherein the hydrolysis or alcoholysis is carried out under acid catalysis or basic catalysis.

30. The process according to claim 16, wherein the hydrolysis or alcoholysis is carried out at a temperature T3 of below 120° C. and above 40° C., and during a reaction t₃ of below 20 hours and above 1 hour.
